(19) United States
(12) Patent Application Publication
(10) Pub. No.: US 2024/0353427 A1
(43) Pub. Date: Oct. 24, 2024

(54) DIAGNOSTIC METHOD OF BARRET'S OESOPHAGUS

(71) Applicant: Cyted Ltd, Cambridge (GB)

(72) Inventors: Marcel GEHRUNG, Cambridge (GB); Anna Maria TSAKIROGLOU, Cambridge (GB)

(21) Appl. No.: 18/684,936

(22) PCT Filed: Aug. 18, 2022
(86) PCT No.: PCT/GB2022/052146

§ 371 Date: Feb. 20, 2024

(30) Foreign Application Priority Data
Aug. 20, 2021 (GB) 2111998.7

(51) Int. Cl. G01N 33/68; G06T 7/00; G16H 30/40; G16H 50/20

(52) U.S. Cl. CPC G01N 33/6893; G06T 7/0012; G16H 30/40; G16H 50/20; G01N 2800/52; G06T 2207/20084; G06T 2207/30024

(57) ABSTRACT

A method useful in the diagnosis of Barrett's oesophagus in a subject, comprises:

a) providing a sample of cells collected from the surface of the oesophagus of the subject;
b) detecting a biomarker in the sample using a biochemical assay;
c) determining a parameter representative of the proportion of cells in the sample that comprise the biomarker;
d) comparing the parameter calculated in step c) to at least one pre-determined cut-off value indicative of Prague stage; and
e) providing an output based on the comparison.

Such methods are useful in selecting subjects for therapy and in methods of treating Barrett's oesophagus. The methods of the invention may be computer-implemented and an additional aspect of the invention is a computer program product storing computer executable instructions for performing computer implemented steps of the method of the invention.

### FIELD OF THE INVENTION

The present invention relates to a diagnostic method useful in the diagnosis of the Prague stage of Barrett's oesophagus.

### BACKGROUND OF THE INVENTION

Barrett's oesophagus is a condition in which there is an abnormal change in the mucosal cells lining the lower portion of the oesophagus, from normal stratified squamous epithelium to simple columnar epithelium with interspersed goblet cells that are normally present only in the small intestine, and large intestine. This change is a premalignant condition because it is associated with a high incidence of further transition to oesophageal cancer.

Barrett's oesophagus is diagnosed by endoscopy, specifically by observing the characteristic appearance of this condition by direct inspection of the lower oesophagus, followed by microscopic examination of tissue from the affected area obtained from biopsy. The cells of Barrett's oesophagus are classified into four categories: nondysplastic, low-grade dysplasia, high-grade dysplasia, and frank carcinoma. High-grade dysplasia and early stages of adenocarcinoma may be treated by endoscopic resection or radiofrequency ablation. Later stages of cancer may be treated with surgical resection. Those with nondysplastic or low-grade dysplasia are usually managed by annual observation with endoscopy, or treatment with radiofrequency ablation.

The guidelines for the diagnosis and management of Barrett's oesophagus can be found in Rebecca C Fitzgerald, Massimiliano di Pietro, Krish Ragunath, et al. Management of Barrett's oesophagus guidelines on the diagnosis and British Society of Gastroenterology (https://www.bsg.org.uk/wp-content/uploads/2019/12/BSG-guidelines-on-the-diagnosis-and-management-of-Barretts-oesophagus.pdf) updated in the Revised British Society of Gastroenterology recommendations on the diagnosis and management of Barrett's oesophagus with low-grade dysplasia. Both documents are hereby incorporated herein by reference. There are equivalent guidelines in the US (AGA).

The Prague C and M stage is commonly used in the clinical management of Barrett's Oesophagus to indicate disease severity and has been validated as a prognostic biomarker of disease¹. Its assessment is done during endoscopy and consists of two components; C is the distance between the proximal cardial notch and the proximal limit of the circumferential Barrett's segment, while M is the distance between the proximal cardial notch and the longest tongue of Barrett's. Both C and M are measured in cm. By way of example, if the circumferential segment (C) is 3 cm and the tongue an additional 2 cm, so that M is 5 cm (3 cm circumferential+2 cm tongue=5 cm maximum Barrett's extent, M), the length of the Barrett's is reported as C3M5.

The Cytosponge procedure is quickly gaining traction as an alternative to endoscopy that minimises the invasiveness of the procedure and does not require anaesthesia. Cytosponge is a ‘pill on a string’ for collecting oesophageal cells. The samples may be analysed using immunohistochemical staining for a biomarker such as trefoil factor 3 (TFF3). This is a biomarker for Barrett's and when identified in the cells contained in the sample during examination under a microscope, it indicates that Barrett's is present. This assay can be used to diagnose Barrett's Oesophagus with confidence; however, it cannot provide an estimate for the severity of the disease, nor assess patient prognosis. This is needed to stratify patients and to ensure they receive the necessary follow-up.

There is therefore a need for a method for non-endoscopically estimating the Prague-stage of Barret's oesophagus, that works with both the ‘pill on a string’ procedures, but also other non-endoscopic assays.

### SUMMARY OF THE INVENTION

It has surprisingly been found that Prague stage can be clinically estimated by non-endoscopic means, more specifically by taking a sample of cells from the surface of the oesophagus and by measuring the proportion of cells in the sample that comprise a particular biomarker (or panel of biomarkers) of interest. This ratio can be used as a proxy biomarker for Prague stage. This is very useful clinically because currently there is no way to estimate Prague stage without an invasive endoscopy.

The present invention is based on the detailed analysis of clinical samples, as described in the Example. In the experiments and analysis described in the Example, the inventors analysed immunohistochemically stained (using the known biomarker TFF3) tissue section samples obtained from the surface of the oesophagus and explored the size of stained area as a potentially useful biomarker. It was demonstrated that extensive biomarker expression correlated with high Prague C and M stages, indicating that a Prague stage could be clinically estimated from a tissue section sample (e.g. collected using cytosponge), without invasive endoscopy. The utility of this tool was tested as a screening tool to prioritise high-risk patients for more frequent follow-up.

The analysis of the clinical samples can be done by hand (by a pathologist, for example), but it may be more useful to have the analysis conducted using a computer. This has been demonstrated experimentally in the Example.

Therefore, in a first aspect, a method useful in the diagnosis of Barrett's oesophagus in a subject, comprises:
a) providing a sample of cells collected from the surface of the oesophagus of the subject;
b) detecting a biomarker in the sample using a biochemical assay;
c) determining a parameter representative of the proportion of cells in the sample that comprise the biomarker;
d) comparing the parameter calculated in step c) to at least one pre-determined cut-off value indicative of Prague stage; and
e) providing an output based on the comparison.

In a second aspect, a computer-implemented method useful in the diagnosis of Barrett's oesophagus in a subject, comprises:
a) receiving image data obtained from the analysis of a tissue section sample derived from the oesophagus of the subject;
b) processing the image data to
   i. classify areas of the tissue section based on image recognition as either type A or type B;
   ii. determine the proportion of type A and/or type B areas relative to the total area of the tissue sample;
   iii. compare the proportion calculated in step ii) to at least one pre-determined cut-off value; wherein the or each cut-off value is indicative of Prague-stage of Barret's oesophagus; and
c) providing an output based on the comparison.

In a third aspect, a computer program product stores computer executable instructions for performing the computer implemented steps of the method defined above.

In a fourth aspect, the present invention is a method for treating Barret's oesophagus, wherein the patient has been selected for treatment by carrying out the method defined above.

In a fifth aspect, a PPI or an NSAID drug, is useful in the therapy of Barrett's oesophagus in a subject, wherein the subject has been selected for therapy by carrying out the method defined above.

### DESCRIPTION OF THE DRAWINGS

FIG. 1 is a whole slide image (WSI) of a Cytosponge section stained with TFF3.

FIG. 2 shows the receiver operating characteristic curve (ROC) for the task of identifying TFF3 positive tiles automatically.

FIG. 3 shows the correlation between Prague C and M lengths (in cm) with the predicted ratio of TFF3 positive tiles.

FIG. 4 shows the ROC and precision/recall curve for the classification task of assigning patients as having stage at least C3.

### DESCRIPTION OF THE PREFERRED EMBODIMENTS

The present invention is useful in the diagnosis and/or management of Barrett's oesophagus. In some embodiments, the subject has been identified as being at risk of developing Barrett's oesophagus and/or oesophageal cancer. They may also have been selected as part of routine screening. For example, all males over 55 might be screened routinely using the method of the invention.

In some embodiments, the subject has one or more risk factors for oesophageal cancer and/or Barret's oesophagus. These may be selected from:
a) being age 55 or over;
b) being a man;
c) being a smoker;
d) being an alcohol drinker;
e) having gastroesophageal reflux disease;
f) being obese;
g) suffering from achalasia;
h) having a history of certain other cancers; and/or
i) suffering from Tylosis or Plummer-Vinson syndrome.

In some embodiments of the present invention, subjects with gastroesophageal reflux disease may be selected for the diagnostic test of the invention. Those patients may be at particularly high risk of developing Barrett's oesophagus.

### Providing the Cell Sample

Any cell sampling device that collects cells from the surface of the oesophagus may be used in a method of the invention. It may be beneficial to use a ‘pill on a string’ device, where the sample is provided by retrieving a swallowable device from the subject that has been swallowed by the subject, wherein the device comprises an abrasive material configured to collect the cells.

One such cell sampling device is EsoCheck. Another suitable cell sampling device is Cytosponge. Cytosponge consists of a small gelatin capsule. This contains a compressed spherical polyester sponge which is attached to string. The capsule is swallowed and after 5 minutes the capsule dissolves allowing the sponge to expand. Using the string, a nurse then pulls the sponge from the stomach through the oesophagus and out of the mouth. As it travels up the oesophagus it collects cells including some from Barrett's if it is present.

### Detecting the Biomarker

The biomarker is detected using a biochemical assay. Suitable biochemical assays are known, and they are able to differentiate cells that comprise the biomarker and cells that do not comprise the biomarker. Preferably, the biomarker can be detected by immunological means, such that the assay is able to differentiate between cells that express the biomarker and cells that do not express the biomarker. The biomarker may be a methylation biomarker, such that it is detectable by DNA methylation analysis. Suitable methods and procedures for these techniques will be known to the skilled person.

Any biomarker for Barrett's oesophagus may be used in a method of the invention. It may be appropriate to use more than one biomarker in a method of the invention, such as by using a combination or a panel of biomarkers.

In some embodiments, the detecting the biomarker is via immunohistochemistry. Suitable techniques will be known to the skilled person and include fluorescence techniques and staining techniques. Preferably, the detecting of the biomarker comprises immunohistochemically staining a tissue section of the sample. A suitable technique can be selected based on the chosen biomarker.

Immunohistochemistry techniques are well known to the skilled person. They involve creating tissue sections (the samples may be formed by clotting), for example sections of 5 micrometer thickness. The staining procedure may be performed using the Dako EnVision+System (DakoCytomation, Ely, UK) or BenchMark ULTRA (Roche). Briefly, non-specific binding may be blocked by incubation in 5% bovine serum albumin (BSA) in Tris-buffered saline (TBS)—Tween 0.05% for 1 h and endogenous peroxidises may be blocked with the hydrogen peroxide provided with the kit. Tissue sections may be incubated with the primary antibody. A mean of the extent and intensity may be generated for each biopsy, reviewed at high magnification (6400), to generate an overall score for each slide. A suitable technique is described in Gut 2009;58:1451-1459. doi: 10.1136/gut.2009.180281, which is incorporated herein by reference.

Based on immunohistochemical staining, a specific area may be designated as biomarker positive or biomarker negative.

For use in a method of the invention, suitable biomarkers are shown below (with the Gene bank accession numbers):

| | | | | |
|---|---|---|---|---|
| Mcm2 | NM_004526 | | | |
| TFF3 | NM_003226 | | | |
| ABP 1 | NM_001091 | | | |
| DDC | NM_000790 | NM_001082971 | | |
| HOXC 10 | NM_017409 | | | |
| KCNE3 | NM_005472 | | | |
| IAMC2 | NM_005562 | NM_018891 | | |
| MUC 13 | NM_033049 | | | |
| MUC 17 | NM_001040105 | | | |
| NMUR2 | NM_020167 | | | |
| PIGR | NM_002644 | | | |
| TSPAN1 | NM_005727 | | | |
| HOXB5 | NM_002147 | | | |
| RNF217 | NM_152553 | | | |
| CCL28 | NM_148672 | | | |
| AGR3 | NM_176813 | | | |
| CFIR | NM_000492 | | | |
| PAQR5 | NM_001104554 | NM_017705 | | |
| BNIP3 | NM_004052 | | | |
| GOLM1 | NM_177937 | | | |
| PLA2G10 | NM_003561 | | | |
| KCNK5 | NM_003740 | | | |
| MLSTD1 | NM_018099 | | | |
| SLC16A7 | NM_004731 | | | |
| NFE2L2 | NM_006164 | | | |
| CGNL1 | NM_032866 | | | |
| CALML4 | NM_001031733 | NM_033429 | | |
| ACSL5 | NM_016234 | NM_203380 | NM_203379 | |
| KRT8 | NM_002273 | | | |
| TMC7 | NM_024847 | | | |
| FAT | NM_005245 | | | |
| CES3 | NM_024922 | | | |
| SLC7A7 | NM_003982 | | | |
| REG4 | NM_032044 | | | |
| CATSPERB | NM_024764 | | | |
| TSPAN3 | NM_005724 | NM_198902 | | |
| SLC37A1 | NM_018964 | | | |
| GPRC5A | NM_003979 | | | |
| GPT2 | NM_133443 | | | |
| PAIP2B | NM_020459 | | | |
| TRIM29 | NM_012101 | | | |
| IL18 | NM_001562 | | | |
| HSD17B 11 | NM_016245 | | | |
| GSDML | NM_001042471 | NM_018530 | | |
| TACSTD1 | NM_002354 | | | |
| HSD17B2 | NM_002153 | | | |
| KRT7 | NM_005556 | | | |
| CLIC6 | NM_053277 | | | |
| ATP2C2 | NM_014861 | | | |
| HEPH | NM_014799 | NM_138737 | | |
| TPD52L1 | NM_003287 | NM_001003395 | NM_001003396 | NM_001003397 |
| HOXB6 | NM_018952 | | | |
| PLS1 | NM_002670 | | | |
| IL1RN | NM_173841 | NM_173842 | NM_173843 | NM_000577 |
| NT5E | NM_002526 | | | |
| CAB39L | NM_001079670 | NM_030925 | | |
| S100A14 | NM_020672 | | | |
| GDA | NM_004293 | | | |
| TRIM31 | NM_007028 | | | |
| ARPC1B | NM_005720 | | | |
| SLC16A1 | NM_003051 | | | |
| TMC5 | NM_024780 | NM_001105248 | NM_001105249 | |
| C PEB2 | NM_182646 | NM_182485 | | |
| LOC93432 | ENST00000397504 | | | |
| F5 | NM_000130 | | | |
| VLDLR | NM_003383 | NM_001018056 | | |
| GCNT3 | NM_004751 | | | |
| MBOAT2 | NM_138799 | | | |
| CPS1 | NM_001875 | | | |
| GALM | NM_138801 | | | |
| DGKD | NM_152879 | NM_003648 | | |
| FAM102B | NM_001010883 | | | |
| LYN | NM_002350 | | | |
| SFN | NM_006142 | | | |
| GALNT7 | NM_017423 | | | |
| EMP1 | NM_001423 | | | |
| CSTB | NM_000100 | | | |
| RHOC / | NM_001042678 | NM_175744 | NM_001042679 | |
| FLJ14959 | AK027865 | | | |
| SNRPN | NR_001294 | | | |
| ANKS4B | NM_145865 | | | |
| PCLKC | NM_017675 | | | |
| ADH7 | NM_000673 | | | |
| LYZ | NM_000239 | | | |
| S100A16 | NM_080388 | | | |
| SLC6A20 | NM_020208 | NM_022405 | | |
| SCNN1G | NM-001039 | | | |
| HKDC1 | NM_025130 | | | |
| SLC7A2 | NM_001008539 | NM_003046 | | |
| SPG20 | NM_015087 | | | |
| 37681 | NM_178450 | | | |
| FGFBP1 | NM_005130 | | | |
| CA9 | NM_001216 | | | |
| RDX | NM_002906 | | | |
| SAMD9 | NM_017654 | | | |
| SERPINB5 | NM_002639 | | | |
| NMU | NM_006681 | | | |
| CLRN3 | NM_152311 | | | |
| SLC9A4 | NM_001011552 | | | |
| VTCN1 | NM_024626 | | | |
| LOC339977 | NM_001024611 | | | |
| FUT9 | NM_006581 | | | |
| GALNT5 | NM_014568 | | | |
| NR5A2 | NM_205860 | NM_003822 | | |
| OLFM4 | NM_006418 | | | |
| LY75 | NM_002349 | | | |
| SCPEP1 | NM_021626 | | | |
| TACSTD2 | NM_002353 | | | |
| MYO1A | NM_005379 | | | |
| VIL1 | NM_007127 | | | |
| SLC28A2 | NM_004212 | | | |
| DPP4 | NM_001935 | | | |
| AZGP1 | NM_001185 | | | |
| CDH17 | NM_004063 | | | |
| NPNT | NM_001033047 | | | |
| ALDH1A1 | NM_000689 | | | |
| ATP13A4 | NM_032279 | | | |
| ATP7B | NM_000053 | NM_001005918 | | |
| IL2RG | NM_000206 | | | |
| POSTN | NM_006475 | | | |
| FCGBP | NM_003890 | | | |
| GPA33 | NM_005814 | | | |
| DSC 2 | NM_024422 | NM_004949 | | |
| COL6A3 | NM_057167 | NM_057165 | NM_057164 | NM_004369 |
| VNN1 | NM_004666 | | | |
| SLP1 | NM_003064 | | | |
| AIM1 | NM_001624 | | | |
| PRKAA2 | NM_006252 | | | |
| GUCY2C | NM_004963 | | | |
| PI3 | NM_002638 | | | |
| TIMP1 | NM_003254 | | | |
| APOL1 | NM_003661 | NM_145343 | | |
| ANPEP | NM_001150 | | | |
| SLC34A2 | NM_006424 | | | |
| DMBT1 | NM_007329 | NM_004406 | NM_017579 | |
| RGS2 | NM_002923 | | | |
| PAPSS2 | NM_004670 | NM_001015880 | | |
| BCMO1 | NM_017429 | | | |
| ADH6 | NM_000672 | NM_001102470 | | |
| TM4SF20 | NM_024795 | | | |
| CHST5 | NM_024533 | | | |
| HHLA2 | NM_007072 | | | |
| FABP1 | NM_001443 | | | |
| SNORD116-21 | NR_003335 | NR_003106 | | |
| MYO7B | NM_001080527 | | | |
| MIA | NM_006533 | | | |
| MEP1A | NM_005588 | | | |
| SLC3A1 | NM_000341 | | | |
| PLAC8 | NM_016619 | | | |
| TFPI | NM_006287 | NM_001032281 | | |
| PGC | NM_002630 | | | |
| MUC 2 | ENST00000361558 | | | |
| LIPF | NM_004190 | | | |
| FABP2 | NM_000134 | | | |
| SI | NM_001041 | | | |
| SLC17A4 | NM_005495 | | | |
| GSTA1 | NM_145740 | | | |
| PDZK1 | NM_002614 | | | |
| RAB3B | NM_002867 | | | |
| REG1A | NM_002909 | | | |
| SPINK4 | NM_014471 | | | |
| CXCL1 | NM_001511 | | | |
| GKN1 | NM_019617 | | | |
| BTNL3 | NM_197975 | | | |
| ADH4 | NM_000670 | | | |
| ALDOB | NM_000035 | | | |
| CXCL2 | NM_002089 | | | |
| SLC26A3 | NM_000111 | | | |
| MMP7 | NM_002423 | | | |
| UPK1B | NM_006952 | | | |
| MEP1B | NM_005925 | | | |
| CA1 | NM_001738 | | | |
| PRSS7 | NM_002772 | | | |

Preferably, the biomarker is selected from:

TFF3, Mcm2, ABP 1, DDC, HOXC 10, KCNE3, IAMC2, MUC 13, MUC 17, NMUR2, PIGR, TSPAN1, HOXB5 or any combination thereof.

More preferably, the biomarker is TFF3.

The biomarker may be a methylation biomarker, preferably selected from mCCNA1, and mVIM.

The present invention involves determining a parameter representative of the proportion of cells in the sample that comprise a biomarker of interest and comparing that parameter to pre-determined cut-off value.

In a preferred embodiment, the cut-off values are established through analysis of cohort data obtained from subjects with a known Prague stage that has been determined by endoscopy and known parameters representative of the proportion of cells that express the biomarker.

In some embodiments, to select a cut-off, development and validation datasets are identified using bootstrapping. Then ROC and precision/recall curves can be plotted for the development dataset. The optimal cut-off can then be identified as the cut-off that resulted in a desired level of sensitivity, specificity, positive predictive value (PPV), negative predictive value (NPV), or a combination thereof. For example, a cut-off may be identified that results in high sensitivity, without significantly compromising PPV, in the development dataset. This cut-off may be subsequently applied and validated on the validation dataset. The process may be repeated (for example up to 10 times) for different random development/validation splits of the dataset.

The cut-off value may be any value identified in tables 2, 3 or 4 of the Example.

The sensitivity for the method of the invention of a particular cut off may be greater than or equal to 0.85, 0.9, 0.91 or 0.92.

The PPV for the method of the invention of a particular cut off may be greater than or equal to 0.7, 0.75, 0.8, 0.85, 0.9 or 0.95.

The specificity for the method of the invention of a particular cut off may be greater than or equal to 0.1, 0.15, 0.2, 0.25, 0.3, 0.35, 0.4 or 0.45.

The NPV for the method of the invention of a particular cut off may be greater than or equal to 0.4, 0.45, 0.5, 0.55, 0.6, 0.65, 0.7, 0.75, 0.8, 0.85, 0.9, 0.91, 0.92, 0.95 or 0.6.

The parameter representative of the proportion of cells in the sample may be dependent on the extent of staining or fluorescence (depending on the technique used). This can be used to approximate a ratio of the cells that comprise (for example, that express) the biomarker to the total number of cells in the sample.

Various techniques are available for estimating that ratio and these will be known to the skilled person. For example, in immunohistochemical analysis of a 2D or 3D (preferably a 2D) tissue section, the ratio of cells may be estimated by measuring the extent of staining. The ratio may be approximated by tessellating the tissue section and classifying each tile as biomarker positive or biomarker negative (depending on the level of staining, for example). The parameter representative of the proportion of cells may therefore be the ratio of biomarker positive tiles to the total number of tiles in the section.

In some embodiments, the classification of the tiles may be done by a human, such as by a trained person or by a pathologist. In some embodiments, the classifying is carried out by computer image recognition, preferably using a machine learning model.

A machine learning model suitable for use in the invention may be trained using a plurality of images of tissue sections each having at least one known biomarker positive or biomarker negative tile. Preferably, the machine learning model is a convolutional neural network (CNN) model.

A computer-implemented method according to the present invention may involve processing the image data too classify areas of tissue session based on image recognition as either type A or type B, or “biomarker positive” or “biomarker negative”. This may be achieved by tessellation and classifying the individual tiles. The ratio of the different types of tiles to the total number of tiles may then be calculated.

The type A and/or type B areas may correspond to the presence or absence of a biomarker.

Computer image recognition techniques can be used to identify and categorise tissue samples as either biomarker positive or biomarker negative. For example, a machine learning model, such as a convolutional neural network (CNN) model, can be used to recognise tissue samples that are positive for the biomarker TFF3 (or any other biomarker of interest).

When using a machine learning model to categorise tissue samples, the model must first be trained using ground truth data comprising images of samples that are already known to be either biomarker positive or biomarker negative. In particular, a first set of images (a training set) including samples that are biomarker positive and samples that are biomarker negative may be given as inputs to the machine learning model along with the corresponding positive/negative classification for each image, which may have been determined from e.g. manual inspection of the samples.

The trained model will preferably then be tested by providing it with a second set of images (a validation set, unrelated to the training set) showing samples that are known to be biomarker positive or negative, but this time the model is not provided with the corresponding positive/negative classifications. Provided that the trained model meets a desired accuracy threshold when categorising the images in the validation set, the trained model can then be used to analyse new images of tissue samples that have not previously been categorised.

In some embodiments, the method comprises comparing the parameter to multiple cut-off values, wherein each cut-off value is indicative of a particular Prague-stage of Barret's oesophagus.

Preferably, the Prague stage is At least C1, At least M1, At least C2, At least M2, At least C3, At least M3, At least C1 or M3 or any combination thereof. More preferably, the Prague state is at least C1 or M3, which is recognised as useful in diagnosis in the clinical guidelines.

### The Output of the Method/Test

In some embodiments, the output comprises a risk level associated with Barrett's oesophagus and/or oesophageal cancer for the subject. This risk level may be expressed as a colour, such as green, red or amber.

In some embodiments, the output comprises a clinical recommendation for the subject preferably selected from an endoscopy, drug therapy, endoscopic resection, endoscopic ablation, repeat biomarker testing within a specified time-period or a combination thereof. The output may be any recommendation listed in the clinical guidelines for diagnosis/management of Barrett's oesophagus. These will be known to the skilled person and are referenced herein (and incorporated herein by reference).

Retesting may be recommended within a specified time-period. This time period may be 6 months, 1 year, 2 years, 3 years, 4 years or 5 years.

Drug therapy may be recommended by the output of the test method of the invention. Such drugs are known to the skilled person and referenced in the clinical guidelines. They may comprise an NSAID or a PPI.

The output of the test method of the invention may be the diagnosis of a particular Prague stage of Barret's oesophagus in the subject.

Preferably, the Prague stage is At least C1, At least M1, At least C2, At least M2, At least C3, At least M3, At least C1 or M3 or any combination thereof. More preferably, the Prague state is At least C1 or M3, which is recognised as useful in diagnosis in the clinical guidelines.

The following example illustrates the invention.

### EXAMPLE

### Methods

### Datasets

Two datasets were used in this analysis, as described below. Patients in both datasets had been recruited and participated in the BEST2 study. There was no overlap between Dataset A and Dataset B.

### Dataset A

Dataset A included 80 patients and was used to develop and validate a deep convolutional neural network (CNN) to predict which areas in TFF3 whole slide images contain positive (i.e., stained) cells. A whole slide image (WSI) of a Cytosponge section stained with TFF3 was available for each patient in Dataset A. Polygon annotations were drawn on these WSI by a pathologist, indicating examples of TFF3 positive and TFF3 negative areas. Approximately half of these patients were diagnosed with Barrett's Oesophagus.

### Dataset B

Dataset B included 462 patients who, after undergoing endoscopy, were all diagnosed with Barrett's Oesophagus. Prague C and M stage (in cm) were recorded for these patients.

### Training a Deep CNN to Identify TFF3 Positive Areas

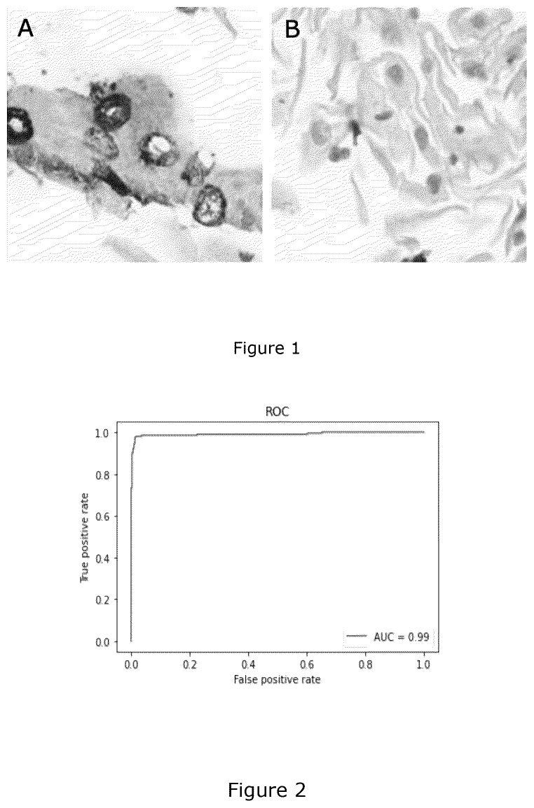

A DenseNet (Dense Convolutional Network) 2 architecture was selected to build an automated model for identification of TFF3 positive areas in WSI. The model was trained and validated on annotated image areas of Dataset A, that were tiled into rectangles of size 500×500 pixels. These tiles were extracted with no overlap, at maximum resolution. From the existing pathologist annotations, each tile had a known label, either “TFF3 positive” or “TFF3 negative”. These labels are considered as the “ground truth”. FIG. 1 shows examples of such tiles. “A” shows the TFF3 positive tile area, featuring goblet cells, indicative of Barrett's Oesophagus, while “B” shows a tile with no TFF3 positive goblet cells.

Each tile image undergoes a series of transformations as it passes through the layers of the network and a predicted output label is generated for each tile. This output is compared to the ground truth and the parameters of the network were updated during training to decrease the prediction error. This error was monitored using a binary cross-entropy loss function. Before training began, the weights of the DenseNet were initialised using pretrained values from a DenseNet-121 model, previously trained on the ImageNet dataset. Only the last 5 layers of the DenseNet were updated during in-house model training. The tile images were resized to 200×200 and pre-processed as required to match the input expected by the pretrained DenseNet model.

From all WSI in Dataset A, 20 were used to train the DenseNet and 40 to validate its performance and fine-tune its hyperparameters (e.g., learning rate, batch size during training). Finally, an additional 20 WSI were used as a test set, to assert that the model can generalise well to previously unseen patients. There was no overlap of patients between the training, validation and test sets.

The CNN was trained for 20 epochs, and the learning rate was decreased by exponential decay, whenever a plateau was reached in the training accuracy. The best model was selected by observing the minimum loss, calculated from the validation set. Multiple training rounds were repeated for different learning rates and batch sizes, to fine-tune the optimal hyperparameters than minimised the validation loss. A Bayesian optimisation strategy was used during hyperparameter tuning.

### Evaluating Correlation with Praque Stage

Once the CNN model demonstrated satisfactory ability to identify automatically TFF3 positive tiles in WSI, it was applied on all patients of Dataset B, to predict the number of TFF3 positive tiles for each patient. The ratio of positive to all tissue tiles can be considered as a proxy of the extent of TFF3 stained area.

The relationship between the extent of TFF3 positive staining and Prague stage was first evaluated by considering the level of correlation with C and M lengths individually in the entire Dataset B (N=462). C and M in this case were treated as continuous variables. Spearman's correlation coefficient was calculated, and significance was assessed at level α=0.01.

Next, we evaluated the ability of this biomarker to predict high Prague stage. Various cut-offs have clinical significance and could be used to define what is considered high Prague stage; Stage at least C1M1 is considered true Barrett's, as opposed to focal intestinal metaplasia at the junction. Length of Barrett's longer than 3 cm is recommended for shorter surveillance intervals according to guidelines by the British Society of Gastroenterologyl and the American College Guidelines,³ while length longer than 6 cm has been correlated with even higher risk of developing oesophageal cancer.4 Thus, our analysis tested the biomarker's ability to identify various subsets of patients by examining a range of cut-offs: at least C1 or M1, at least C3 or M3, and at least C6 or M6.

### TFF3 Staining as a Screening Tool to Identify High-Stage Patients

If we consider an assay where a positive result is having high stage and a negative result is having low stage, good assay performance in this setting translates to high sensitivity in identifying positive patients (i.e., high stage). In the analysis that follows, bootstrapping is performed on Dataset B to identify a suitable range of cut-offs for the ratio of TFF3 positive tiles.

In bootstrapping, Dataset B was randomly split in half repeatedly (10 iterations) to form development and validation datasets. Cut-offs were selected to identify high stage patients with optimal sensitivity in the development datasets and performance was evaluated on the validation datasets. In this way, a range for the optimal cut-off and the performance metrics were identified.

### RESULTS

### CNN Tile-Level Performance on Dataset A

The developed model achieved excellent performance in the validation set (N=40, see FIG. 2) and was able to identify TFF3 positive tiles in the test set (N=20) with a sensitivity of 0.917 and precision of 0.925.

FIG. 2 depicts the receiver operating characteristic curve (ROC) for the task of identifying TFF3 positive tiles automatically. The area under the curve (AUC) measures the accuracy of classification. For the trained CNN model, AUC=0.99.

Correlation between the ratio of TFF3 positive tiles and C/M lengths in Dataset B

Based on its high precision and sensitivity, this model was considered a suitable alternative to manual assessment for estimating the extent of TFF3 expression and was subsequently applied on all patients of Dataset B to obtain the ratio of TFF3 positive tiles per patient.

TABLE 1
Spearman's correlation coefficient to assess the relationship
between Prague stage C and M lengths and the ratio of TFF3
positive tiles in all 462 patients of Dataset B.

| Correlation of TFF3 | | |
|---|---|---|
| expression with Prague | | |
| stage | Spearman's rho | P value |
| Prague C | 0.54 | <10⁻³⁶ |
| Prague M | 0.59 | <10⁻⁴⁴ |

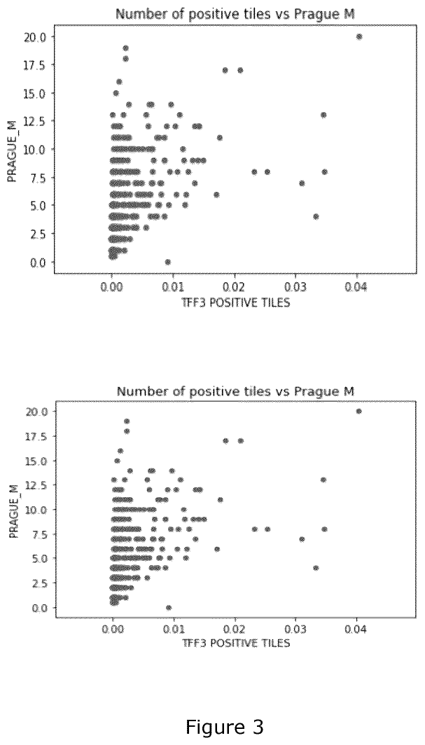

Table 1 and FIG. 3 show a very significant positive correlation between the ratio of predicted TFF3 positive tiles and Prague C and M lengths (p<10-36 and p<10-44, respectively). FIG. 3 depicts the correlation between Prague C and M lengths (in cm) with the predicted ratio of TFF3 positive tiles.

### Prevalence of High Prague Stage

The extent of TFF3 expression was subsequently evaluated as a biomarker to predict high Prague stage. Tables 2 shows how many patients had stages of at least 1 cm, 3 cm and 6 cm in Dataset B

A very small number of patients had M1 stage less than M1, indicating focal intestinal metaplasia at the junction, instead of true Barrett's is a very rare event.

TABLE 2
Prevalence of high Prague stage in the Dataset B.

| | | | | | | At | |
|---|---|---|---|---|---|---|---|
| At | At | At | At | At | At | least | |
| least | least | least | least | least | least | C1 or | |
| M1 | M3 | M6 | C1 | C3 | C6 | M3 | Total |
| 451 | 336 | 339 | 193 | 156 | 103 | 375 | 462 |

### Ratio of TFF3 Positive Tiles as a Biomarker of High Prague Stage

The ratio of TFF3 positive to all tissue tiles was evaluated as a biomarker to predict high Prague stage. To assess the performance of this biomarker, a cut-off was selected to optimise its sensitivity for the task of identifying high stage patients that need frequent follow-up. In this assay, a positive result corresponds to a high stage. Patients with biomarker values above the cut-off are predicted to have high stages.

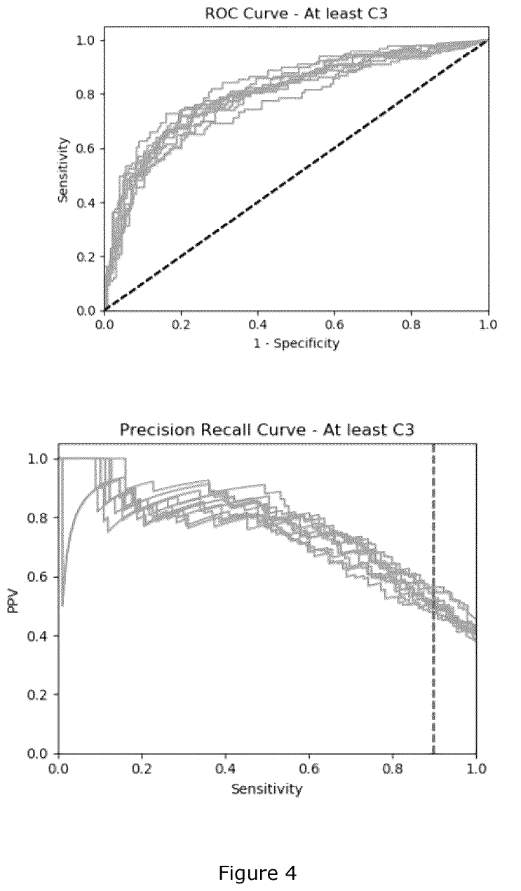

To select a cut-off, development and validation datasets were identified using bootstrapping. Then ROC and precision/recall curves were plotted for the development dataset (FIG. 4). The optimal cut-off was identified as the cut-off that resulted in high sensitivity, without significantly compromising PPV, in the development dataset. This cut-off was subsequently applied and validated on the validation dataset. The process was repeated for 10 different random development/validation splits of the dataset.

FIG. 4 shows the ROC and precision/recall curve for the classification task of assigning patients as having stage at least C3. Curves are plotted for the development dataset. The dotted line in the ROC plot represents the diagonal. The dotted line in the precision/recall curve shows the sensitivity at the optimal selected cut-off. A separate curve is plotted for each bootstrapping iteration.

Table 3 shows the selected optimal cut-offs (average and standard deviation from bootstrapping iterations) on the ratio of TFF3 positive tiles.

TABLE 3
Optimal biomarker cut-offs (average and standard
deviation), selected by bootstrapping.

| | | | | | | | At |
|---|---|---|---|---|---|---|---|
| | At | At | At | At | At | At | least |
| | least | least | least | least | least | least | C1 or |
| | C1 | M1 | C3 | M3 | C6 | M6 | M3 |
| Opti- | 0.0034 | 0.0027 | 0.0126 | 0.0070 | 0.0231 | 0.0164 | 0.0037 |
| mal | | | | | | | |
| cut- | | | | | | | |
| off* | | | | | | | |
| STD | 0.0009 | 0.0001 | 0.0027 | 0.0016 | 0.0045 | 0.0041 | 0.0010 |

*The optimal cut-off is presented as the % of positive TFF3 tiles to all tiles belonging to tissue areas.
PPV: Positive prognostic value;
STD: standard deviation

Table 4 shows the performance of the biomarker on the validation set, using the optimal cut-offs.

TABLE 4
Performance metrics (average and standard deviation) of the biomarker
at the selected cut-off. Data shown for the validation set.

| | | | | | | | At |
|---|---|---|---|---|---|---|---|
| | At | At | At | At | At | At | least |
| | least | least | least | least | least | least | C1 or |
| Metric | C1 | M1 | C3 | M3 | C6 | M6 | M3 |
| Sensitivity | 0.922 | 0.913 | 0.919 | 0.909 | 0.927 | 0.933 | 0.924 |
| PPV | 0.759 | 0.982 | 0.512 | 0.797 | 0.327 | 0.455 | 0.849 |
| Specificity | 0.215 | 0.358 | 0.358 | 0.385 | 0.418 | 0.397 | 0.313 |
| NPV | 0.52 | 0.096 | 0.862 | 0.623 | 0.951 | 0.922 | 0.501 |
| Sensitivity | 0.031 | 0.015 | 0.031 | 0.032 | 0.035 | 0.035 | 0.024 |
| STD | | | | | | | |
| PPV STD | 0.018 | 0.006 | 0.022 | 0.022 | 0.03 | 0.035 | 0.018 |
| Specificity | 0.042 | 0.131 | 0.038 | 0.053 | 0.051 | 0.063 | 0.055 |
| STD | | | | | | | |
| NPV STD | 0.096 | 0.041 | 0.04 | 0.075 | 0.023 | 0.032 | 0.079 |

PPV: positive predictive value; NPV: negative predictive value. Sensitivity >0.9 is shown in bold; STD: standard deviation

While the biomarker results in good sensitivity (>90%) across all Prague stage cut-offs, it is generally more precise (higher PPV) in predicting the length of M segments, as opposed to C segments.

### Discussion

The extent of TFF3 staining in Cytosponge samples could be used as a screening tool to identify high stage patients. These are patients at high risk of developing Oesophageal cancer and would benefit from prioritisation in clinical management and shorter surveillance intervals. In this setting, the biomarker-based assay would not need to be extremely precise but would need to be very sensitive in identifying all patients that need urgent endoscopy and/or more frequent follow up. High sensitivity is needed to ensure that no patient in need of escalated clinical intervention is missed.

Our results show that a biomarker based on the ratio of TFF3 positive tiles is able to identify patients with advanced Prague stage with high sensitivity and adequate precision. Good performance is maintained across a range of different Prague stage cut-offs, which would allow tailoring of interventions to different patient subsets. For example, this biomarker could be used to prioritise endoscopy for patients with maximal length (M) longer than 3 cm, which qualify for frequent follow-up, according to British Society of Gastroenterologyl. Use of this biomarker would pick out 90.9% of these patients with 79.7% precision (Table 4). Even higher sensitivity could be achieved for selecting patients with more advanced C and M Barrett's segments, longer than 6 cm (≥92%). These patients have an elevated risk of cancer,⁴ therefore their early identification is critical.

A potential alternative use of this biomarker would be as a precise indicator of Prague stage that would render the need for endoscopy redundant for a subset of patients with low stage. If this biomarker accurately identifies patients with low stage, then endoscopy may no longer be necessary for these patients. In this setting, the biomarker-based assay would not need to exhaustively identify all low stage patients but would need to be very confident that patients predicted as low stage are truly low stage, as these patients would not receive further endoscopy for confirmation. The high negative predictive value (NPV>0.8) obtained by some of the models described in 3.3 (Table 4) show that this approach could merit further exploration.

In the proposed biomarker, the extent of TFF3 staining expression is estimated automatically, using a machine learning model to enumerate TFF3 positive tiles in whole slide images. The significant benefit of this approach is that it is fast and quantitative, while manual estimation of the TFF3 stained area by a pathologist could be cumbersome and error prone.

Overall, the evidence suggests that the extent of TFF3 staining expression can provide a useful biomarker to screen patients with high Prague C and M stages solely from Cytosponge samples and prioritise suitable clinical interventions.

### REFERENCES (All INCORPORATED HEREIN BY REFERENCE)

1. Fitzgerald R C, Di Pietro M, Ragunath K, et al. British Society of Gastroenterology guidelines on the diagnosis and management of Barrett's oesophagus. Gut. 2014;63 (1): 7-42.
2. Huang G, Liu Z, Van Der Maaten L, Weinberger KQ. Densely connected convolutional networks. In: Proceedings of the IEEE Conference on Computer Vision and Pattern Recognition; 2017:4700-4708.
3. Shaheen N J, Falk G W, lyer P G, Gerson L B. ACG clinical guideline: diagnosis and management of Barrett's esophagus. Off J Am Coll Gastroenterol ACG. 2016;111 (1): 30-50.
4. Parasa S, Vennalaganti S, Gaddam S, et al. Development and validation of a model to determine risk of progression of Barrett's esophagus to neoplasia. Gastroenterology. 2018;154 (5): 1282-1289.

1. A method useful in the diagnosis of Barrett's oesophagus in a subject, comprising:
a) providing a sample of cells collected from the surface of the oesophagus of the subject;
b) detecting a biomarker in the sample using a biochemical assay;
c) determining a parameter representative of the proportion of cells in the sample that comprise the biomarker;
d) comparing the parameter calculated in step c) to at least one pre-determined cut-off value indicative of Prague stage; and
e) providing an output based on the comparison.

2. The method according to claim 1, wherein the detecting the biomarker comprises using an immunoassay or by using DNA methylation analysis.

3. The method according to claim 2, wherein the detecting the biomarker comprises immunohistochemically staining a tissue section of the sample, and wherein the parameter representative of the proportion of cells is based on the extent of the staining.

4. The method according to claim 3, wherein the ratio of the stained area to the total area of the tissue section is approximated by tessellating the tissue section and classifying each tile as biomarker positive or biomarker negative.

5. The method according to claim 4, wherein the classifying comprises computer image recognition, preferably using a machine learning model.

6. The method according to claim 5, wherein the machine learning model is trained using a plurality of images of tissue sections each having at least one known biomarker positive or biomarker negative tile.

7. The method according to claim 6, wherein the machine learning model is a convolutional neural network (CNN) model.

8. The method according to claim 1, wherein the sample is provided by retrieving a swallowable device from the subject that has been swallowed by the subject, wherein the device comprises an abrasive material configured to collect the cells.

9. The method according to claim 1, wherein the output comprises a risk level associated with Barrett's oesophagus and/or oesophageal cancer for the subject.

10. The method according claim 1, wherein the output comprises a clinical recommendation for the subject preferably selected from an endoscopy, drug therapy, endoscopic resection, endoscopic ablation, repeat biomarker testing within a specified time-period or a combination thereof.

11. A method according to claim 10, wherein the specified time-period is 6 months, 1 year, 2 years, 3 years, 4 years or 5 years.

12. A method according to claim 10, wherein the drug therapy comprises treatment with an NSAID or a PPI.

13. A method according to claim 1 wherein the output is the diagnosis of a particular Prague stage of Barret's oesophagus in the subject.

14. A method according to claim 13, wherein the Prague stage is At least C1, At least M1, At least C2, At least M2, At least C3, At least M3, At least C1 or M3 or any combination thereof.

15. The method according to claim 1, wherein step d) comprises comparing the parameter calculated in step c) to multiple cut-off values, wherein each cut-off value is indicative of a particular Prague-stage of Barret's oesophagus, wherein the Prague stage is preferably as defined in claim 14.

16. A method according to claim 15, wherein the cut-off values are established through analysis of cohort data obtained from subjects with a known Prague stage that has been determined by endoscopy and known parameters representative of the proportion of cells that express the biomarker.

17. A method according to claim 15, wherein the cut-off values are selected according to a desired sensitivity, specificity, positive predictive value or negative predictive value, or any combination thereof, for determining the output.

18. A method according to claim 1, wherein the subject has been identified as being at risk of developing oesophageal cancer.

19. A method according to claim 1, wherein the subject has one or more risk factors for oesophageal cancer and/or Barret's oesophagus, preferably selected from:
a) being age 55 or over;
b) being a man;
c) being a smoker;
d) being an alcohol drinker;
e) having gastroesophageal reflux disease;
f) being obese;
g) suffering from achalasia;
h) having a history of certain other cancers; and/or
i) suffering from Tylosis or Plummer-Vinson syndrome.

20. The method according to any preceding claim 1, wherein the biomarker is selected from TFF3, Mcm2, ABP 1, DDC, HOXC 10, KCNE3, IAMC2, MUC 13, MUC 17, NMUR2, PIGR, TSPAN1, HOXB5 mCCNA1, and mVIM or any combination thereof.

21. The method according to claim 20, wherein the biomarker is TFF3.

22. A method for treating Barrett's oesophagus, comprising:
a) providing a sample of cells collected from the surface of the oesophagus of the subject:
b) detecting a biomarker in the sample using a biochemical assay;
c) determining a parameter representative of the proportion of cells in the sample that comprise the biomarker;
d) comparing the parameter calculated in step c) to at least one pre-determined cut-off value indicative of Prague stage;
e) providing an output based on the comparison;
f) selecting an appropriate treatment based on the output; and
g) administering the treatment to a patient in need thereof.

23. A method according to claim 22, wherein the treatment comprises drug therapy, preferably with a PPI or an NSAID, endoscopic resection, and/or endoscopic ablation.

24. (canceled)

25. A computer-implemented method useful in the diagnosis of Barrett's oesophagus in a subject, comprising:
a) receiving image data obtained from the analysis of a tissue section sample derived from the oesophagus of the subject;
b) processing the image data to
i. classify areas of the tissue section based on image recognition as either type A or type B;
ii. determine the proportion of type A and/or type B areas relative to the total area of the tissue sample;
iii. compare the proportion calculated in step ii) to at least one pre-determined cut-off value; wherein the or each cut-off value is indicative of Prague-stage of Barret's oesophagus; and
c) providing an output based on the comparison.

26. A computer-implemented method according to claim 25, wherein the classifying areas further comprises tessellation of the image followed by classifying the individual tiles as either type A or type B.

27. A computer-implemented method according to claim 26, wherein the determining of the type A and/or type B tiles is approximated by calculating the ratio of the total number of type A and/or type B tiles to the total number of tiles in the image.

28. The method according to claim 25, wherein the classifying comprises computer image recognition, preferably using a machine learning model.

29. The method according to claim 28, wherein the machine learning model is trained using a plurality of images of tissue sections each having at least one known biomarker positive or biomarker negative tile.

30. The method according to claim 29, wherein the machine learning model is a convolutional neural network (CNN) model.

31. The method according to claim 1, wherein the sample is provided by retrieving a swallowable device from the subject that has been swallowed by the subject, wherein the device comprises an abrasive material configured to collect the cells.

32. The method according to claim 25, wherein the output comprises a risk level associated with Barrett's oesophagus and/or oesophageal cancer for the subject or wherein the output comprises a clinical recommendation for the subject preferably selected from an endoscopy, drug therapy, endoscopic resection, endoscopic ablation, repeat biomarker testing within a specified time-period or a combination thereof.

33. A method according to claim 31 wherein the output is the diagnosis of a particular Prague stage of Barret's oesophagus in the subject.

34. A method according to claim 32, wherein the Prague stage is At least C1, At least M1, At least C2, At least M2, At least C3, At least M3, At least C1 or M3 or any combination thereof.

35. A method according to claim 25, wherein the cut-off values are established through analysis of cohort data obtained from subjects with a known Prague stage that has been determined by endoscopy and known parameters representative of a proportion of cells that express a biomarker.

36. The method according to claim 35, wherein the biomarker is TFF3.
